(19) United States
(12) Patent Application Publication
(10) Pub. No.: US 2011/0008410 A1
(43) Pub. Date: Jan. 13, 2011

(54) PHARMACEUTICAL COMPOSITION FOR TREATING MALARIA

(76) Inventor: Joachim Heizmann, Badenweiler (DE)

Correspondence Address: WENDEROTH, LIND & PONACK, L.L.P., 1030 15th Street, N.W.,, Suite 400 East, Washington, DC, 20005-1503, US

(21) Appl. No.: 12/734,959

(22) PCT Filed: Dec. 3, 2008
(86) PCT No.: PCT/EP2008/066669

§ 371 Date: Jul. 28, 2010

(30) Foreign Application Priority Data
Dec. 4, 2007 (CH) 01871/07

(51) Int. Cl. A61K 31/496; A61K 9/48; A61P 33/06

(52) U.S. Cl. 424/436; 514/252.11

(57) ABSTRACT

The invention relates to a pharmaceutical composition in the form of rectal capsules, comprising a combination, effective against malaria parasites, of artemisinine or a derivative of artemisinine, particularly artesunate, with piperaquine or a pharmaceutically acceptable salt thereof, particularly piperaquine tetraphosphate, and having high activity against Plasmodium such as, for example, Plasmodium falciparum.

The present invention relates to a pharmaceutically effective composition, which comprises a combination, effective against parasites of malaria, of artemisinine or a derivative of artemisinine with piperaquine or a pharmaceutically acceptable salt thereof, and which exhibits a high activity against multi-resistant strains of parasites of malaria. In particular the present invention relates to a pharmaceutically effective formulation for rectal application comprising artemisinine or a derivative of artemisinine such as, for example, arteether, artemether, artemisinine or artesunate, in particular artesunate, and piperaquine or a pharmaceutically acceptable salt thereof, in particular piperaquine tetraphosphate.

Malaria in humans is caused by four species of Plasmodium protozoa: Plasmodium falciparum, Plasmodium malariae, Plasmodium vivax and Plasmodium ovale, of which Plasmodium falciparum is most abundant and most virulent. The parasite is transmitted to humans by the bite of the anopheles mosquito.

Patients afflicted with severe malaria caused by Plasmodium falciparum are often unconscious, and peroral treatment is not possible. Parenteral application of medicaments against malaria comes into question only in a hospital or in suitable accommodation under supervision of a medical doctor. In such severe cases a pharmaceutical composition in rectally applicable form may be applied to save lives, and in fact anywhere in the world and in practically all situations. A further advantage of the rectal formulation is the application with children, when peroral application is often associated with problems of swallowing.

In Africa and Southeast Asia approximately one million children die annually of malaria. For the treatment of children in tropical and subtropical countries tablets and injection solutions, mostly present as powder formulation, are available commercially, but neither suppositories nor other rectal formulations. This is mainly linked to the fact that the standard suppositories are instable at higher temperatures, since they are supposed to melt at 37° C. on rectal application. Many subtropical and tropical regions have a warm climate above average and also above average high temperatures, which makes application of suppositories difficult or impossible.

The purpose of the present invention is the provision of a rectal pharmaceutical formulation comprising a combination of artemisinine and/or a derivative of artemisinine such as, for example, arteether, artemether, artemisinine and/or artesunate, in particular artesunate, together with piperaquine or a pharmaceutically effective salt thereof, in particular piperaquine tetraphosphate, as the active ingredient, wherein this formulation shows a satisfactory stability of the active ingredients with adequate shelf life, and comprises a sufficient amount of active ingredients to allow safe treatment of the pathogen Plasmodium in blood.

Rectal pharmaceutical formulations are, for example, suppositories, rectal foams, enema or rectal capsules. Suppositories are rather unsuitable for storing and application in subtropical or tropical regions because of the melting temperature of 37° C., as explained above. Rectal foams and enema are likewise unsuitable for patients under simple circumstances and lack of hygienic facilities because of their complicated method of application. Hence it is proposed to use rectal capsules as rectal forms of application.

The standard precondition for the manufacture of rectal capsules consists in the presence of surface active compounds, in particular surfactants (tensides), but also emulsifiers and optionally wash active compounds to guarantee a sufficient bioavailability of the active ingredients by improvement of the dissemination in the rectum. The studies showed, however, that even after short periods of storage formulations containing artesunate with larger amounts of surfactants are unstable. Further the skilled person in the art would expect based on general knowledge on rectal application that the permeation in the rectum of piperaquine in the form of the phosphate salt as a fourfold protonated active ingredient would be low, and piperaquine would hence be expected to have only low or no therapeutic efficacy. Whereas in the intestine an absorption area of about 100 m² is available for orally applied active ingredients, the absorption area in the rectum is only 0.04 to 0.07 m². Further the rectum only contains 1 to 3 ml mucus to set free the active ingredient, much less than the intestine.

It has now surprisingly been found that rectal capsules comprising a composition containing the active ingredient artemisinine and/or an artemisinine derivative, in particular artesunate, together with piperaquine as the free base or as a salt and additives being inert towards the active ingredients, not only represent a galenically stable formulation providing the required stability criteria concerning the chemical stability of the active ingredients, but also provide a good bioavailability of the active ingredients.

Artesunate is a water soluble derivative of artemisinine. Artemisinine (Quinghaosu) is prepared from the leaves of the Artemisia annua shrub and is a naturally occurring sesquiterpene lactone with an endoperoxide group. Due to the low water solubility of the natural product artemisinine, it has been tried to transform it into different synthetic derivatives in order to get an improved pharmaceutical availability. Such a derivative is also artesunate. Artemisinine and artesunate are efficient active ingredients for the treatment of malaria. Artemisinine preparations are presently the fastest acting compounds against malaria parasites. They show a particular high activity against multiresistant strains of Plasmodium such as Plasmodium falciparum. On application to humans only few side effects and no significant toxicity has been observed. In the body artemisinine and also artesunate are transformed into dihydroartemisinine (DHA, artesol), which is the actual active schizonticide ingredient.

Artesunate is dihydroartemisinine hemisuccinate (C₁₉H₂₈O₈) of the formula

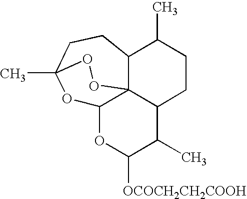

“Artesunate” is used to denote both the free acid and the salts, in particular the sodium salt. Dihydroartemisinine is chemically named 3α,12α-epoxy-3,4,5,5aα,6,7,8aα,9,10,12β,,12a-dodecahydro-10-hydroxy-3β,6a,9β-trimethylpyrano[4,3-j]-1,2-benzodioxepine. Dihydroartemisinine is also known under the name of dihydroquinghaosu. Artesunate and dihydroartemisinine hemisuccinate, respectively, can be prepared, for example, by transforming dihydroartemisinine through acylation into dihydroartemisinine hemisuccinate. Arteether, artemether and artemisinine are known as such.

Piperaquine is a dimeric chloroquinoline derivative substituted by piperazine, which is often applied in China in the form of tablets, with good success in the treatment of malaria caused by Plasmodium falciparum. Whereas artemisinine and derivates of artemisinine act very fast, but lose their activity also fast, piperaquine has an extended activity profile. Piperaquine (C₂₉H₃₂Cl₂N₆) is the compound of the formula

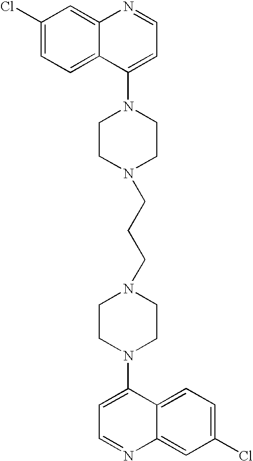

“Piperaquine” denotes both the free acid and the acid addition salts, in particular the (mono)phosphate or the tetraphosphate. Piperaquine is chemically named 7-chloro-4-[4-[3-[4-(7-chloroquinolin-4-yl)piperazin-1-yl]propyl]piperazin-1-yl]quinoline. Piperaquine is also known under the name piperaquinoline. For the manufacture 4,7-dichloroquinoline may first be reacted with piperazine, and then coupled with 1,3-dibromopropane in the presence of a base.

Both piperaquine and dihydroartemisinine are manufactured in particular in China and Vietnam, and sold as tablets. An international patent application WO 2002/026226 (in Chinese) and the corresponding Chinese patent application CN 00113134.6 describe the use of combinations of dihydroartemisinine, piperaquine and optionally trimethoprim.

According to the present invention mixtures of an artemisinine derivative, in particular of artesunate (applied as the sodium salt or, preferably, as the free acid), and piperaquine or a pharmaceutically acceptable salt thereof, in particular the tetraphosphate salt of piperaquine, are used in proportions of 1:0.5 to 1:10, for example in the proportion 1:0.5 to 1:7.5. Preferred is the proportion of artesunate to piperaquine of 1:0.5 to 1:2.5, for example 1:1 or 1:2. For calculation of the proportion the molecular weights are taken for artesunate as the acid (384 g/mol) and for piperaquine as the base (535 g/mol), because the activity of the components in the combination is only marginally dependent on the choice of the counter ion (cation for a salt of artesunate, for example sodium, and anions for a salt of piperaquine, for example the tetraphosphate, i.e. the fourfold protonated piperaquine with four dihydrogen phosphate anions, respectively). The active ingredients may also be present in micronized form.

Preferred are rectal capsules comprising the combination of active ingredients according to the invention, and suitable additives.

Rectal capsules according the present invention are, in particular, hard gelatin capsules or soft gelatin capsules, which contain the composition according to the invention in a pharmaceutically effective amount, and wherein the hard gelatin capsule or the soft gelatin capsule is coated with a lubricating coat consisting of coating materials known as such for rectal capsules. Preferred are soft gelatin capsules.

The present invention relates also to the use of these rectal capsules comprising mixtures of an artemisinine derivative, in particular artesunate, and piperaquine, in particular piperaquine tetraphosphate, in a proportion of 1:0.5 to 1:10 for the treatment of malaria parasites and multiresistant strains of Plasmodium such as Plasmodium falciparum. Preferably soft gelatin capsules with a lubricating coat comprising artesunate and piperaquine tetraphosphate are used in a proportion of 1:0.5 to 1:2.5 and suitable additives.

Additives inert towards the active ingredients are, in particular, waxes, fats and oils, and mixtures thereof. These may be of plant or animal origin, and may also be hydrogenated.

Preferred are waxes, fats and oils of plant origin. Likewise paraffin waxes and paraffin oils may be used.

Waxes are, for example, natural plant waxes, such as carnauba wax, waxes of animal origin, such as yellow or white bee wax, stearin waxes, with melting points (and melting point intervals, respectively) between about 47° C. and about 88° C. Paraffin waxes may also be used, for example, hard paraffins, with melting points between about 54° C. and about 105° C. Such waxes are used mainly as providers of consistency.

Fats are, in general, triglycerides with (C₁₂-C₂₄) fatty acids, predominantly (C₁₆-C₁₈) fatty acids having melting points and melting point intervals, respectively, of between about 28° C. and about 45° C. Hard fats in the form of semisynthetic fats for the manufacture of rectal capsules consist of a mixture of mono-, di- and triglycerides of saturated (C₁₀-C₁₈) fatty acids. Also paraffin fats, that is paraffins with melting points and melting points intervals, respectively, in the indicated regions, may be used.

Oils are, in general, triglyceride of intermediate chain length with (C₈-C₁₆) fatty acids, preferably (C₈-C₁₂) fatty acids, which are liquid at room temperature and having melting points and melting point intervals, respectively, which are between 0° C. and 20° C., preferably at about 0° C. to 10° C. Also paraffin oils, that is paraffins having melting points or melting point intervals, respectively, in the mentioned regions, may be used. Oils are particularly used as solvents or solubilizers, as suspension aids or as emulsifiers. Such fats and/or oils may be prepared, for example, from plant raw materials, such as coconut, palm nuts, olives, canola, or from animal raw materials, such as bovine tallow, lard or blubber, and are optionally hydrogenated.

As further additives, wetting agents may be added in small amounts. Wetting agents may be anionic, cationic, amphoteric or non-ionogenic. Standard wetting agents are, for example, anion active wetting agents, such as alkali or ammonium salts of unsaturated fatty acids, in particular alkali alkylsulfates, e.g. sodium dodecylsulfate, sodium laurylsulfate, sodium cetylstearyl sulfate or sodium docusate, alkali and earth alkali salts of alkyl or arylalkyl sulfonates, and salts of bile acid such as sodium cholate, and acidic saponins. Cationic wetting agents are, for example, quaternary ammonium compounds. Amphoteric wetting agents are, for example, lecithins and betaine derivatives. Non-ionogenic wetting agents are, for example, fatty alcohols, cholesterols, optionally in combination with primary emulsifiers, such as, e.g., emulsifying cetylstearyl alcohol (combination of sodium cetylstearyl sulfate and cetylstearyl alcohol) or cetomacrogol emulsifying wax, cholesterol, partial fatty esters of glycerol, such as glycerol mono-fatty ester, e.g. glycerol monostearate, optionally in combination with hydrophilic emulsifiers, partial fatty acid esters of sorbitan, oxyethylated partial fatty acid esters of sorbitan, other partial fatty acid esters, fatty acid esters of polyoxyethylene, fatty alcohol ethers of polyoxyethylene, fatty acid ester of saccharose, fatty acid esters of polyglycerols, and block copolymers of polyoxyethylene and of polyoxy-propylene. Such surfactants are used, as a rule, in an amount of 10 percent maximum, calculated on filling mass. According to the present invention preferably less than 5 percent or no wetting agents are used.

The weight proportion of the pharmaceutical active ingredients to the additives is preferably 5% to 15%, preferably 7% to 12%, calculated on the total weight of additives. The pharmaceutically active dose for adults is, as a rule, in the region of 75 mg to 600 mg of the combination of active ingredients per capsule, and at about 300 mg on average. A treatment comprises the application of 3 to 5 rectal capsules. Artesunate is applied, for example, in a total dose of 5 mg/kg body weight, and piperaquine in a dose of 8 mg/kg body weight. For application to children rectal capsules are manufactured with lower amounts of the combination of active ingredients, for example comprising between 10 mg and 250 mg of the combination of active ingredients per capsule.

For example, the following proportions of artesunate to piperaquine tetraphosphate have been shown to be advantageous for adults: 100/180 mg, 100/270 mg, 100/360 mg, 200/360 mg, and 50/360 mg.

The consistency of the preparation according to the invention is not critical, because it has almost no influence on the stability of the active ingredient. The consistency of the preparation may be solid, pasty or liquid. Decisive for the selection of the weight proportion of combination of active ingredients to additives and for the selection of the various components of additives are the industrial options for manufacture in the filling procedure of capsules. The suspension may not settle during filling, which means, the uniformity of the content of active ingredients in the preparation of the capsule has to be guaranteed during and after filling. For that purpose the flowability of the preparation usually present as a suspension is adjusted such that the preparation is flowing properly during manufacture, but is, however, a paste or solidifies after manufacture in filled form within the capsule. This is accomplished by processing the preparation under pressure and/or increased temperature during the filling method. It is also possible to add further additives to the preparation, for example, dispersed or highly dispersed aluminium oxide or dispersed or highly dispersed silicium dioxide, which simplify this method of processing.

The quality and type of capsules used correspond to the qualities available in commerce, known as such. The manufacture of the preferred soft gelatin capsules according to the invention is, e.g., described in “Pharmazeutische Technologie”, H. Sucker, P. Fuchs, P. Speiser, Stuttgart 1991, pages 337-347.

The capsule coat serves to improve the lubricating capacity of rectal capsules, and to guarantee the simple introduction in the body. Such lubricating coats are known. Coating materials known as such for rectal capsules are, for example, polyethylene glycols with an average molecular weights of about 1500 to 20000, glycerol monooleate and glycerol dioleate, polyvinyl acetate, and talcum. Hence a composition known as such consists, for example, of 40.5 parts polyethylene glycol with an average molecular weight of about 20000, 17.4 parts polyethylene glycol with an average molecular weight of about 1550, 26.0 parts glycerol monooleate and glycerol dioleate (mixture), 1.2 parts polyvinyl acetate, and 14.9 parts talcum. Capsule coats are described, for example, in “Pharmazeutische Technologie”, H. Sucker, P. Fuchs, P. Speiser, Stuttgart 1991, page 343.

For the manufacture of the preparations according to the invention it is possible, for example, to mix the additives, for example the hard fat, in a heatable and evacuatable suspension process apparatus at melting temperature. Further additives are subsequently added to the melt, for example triglycerides with intermediate chain length, and also the active ingredient, and the melt is processed to give a homogenous mass. The obtained molten suspension is then filtered through a sieve. The filtrate is afterwards degassed by application of vacuum and filled into capsules. The capsules thus obtained are covered with a capsule coat according to a method known as such. It is also possible to use a different customary procedure known in the art.

The following examples explain the invention without limiting it in any way.

### EXAMPLE 1

### Manufacture of a Composition According to the Invention

In a heatable and evacuatable suspension process apparatus made from stainless steel with integrated stirrer, homogenizer and recirculating pump system (Diessel-Werke) 10 kg hard fat are molten at 45° C. Then 30 kg triglycerides of intermediate chain length and thereafter 20 kg artesunate and 36 kg piperaquine tetraphosphate are worked in by stirring. The mass is subsequently homogenized by recycling during 10 minutes. Then the suspension is kept at 40° C. +/−2° C. with moderate stirring, and pressed through a sieve of 400 μm mesh. The filtered suspension is degassed by application of vacuum (residual pressure 0.5 to 0.2 bar, absolute) during 2 to 3 hours.

The obtained mixture is filled into soft gelatin capsules as follows:

(a) To a separate heatable stainless steel vessel with stirrer (Diessel) 40 kg gelatin, 16 kg glycerol (85%) and 28% purified water are added. The mass is molten to a clear melt at 70° C. Gas bubbles are eliminated by slow stirring. To the melt is added a suspension of 0.70 kg titanium dioxide in glycerol (85%) (1 part titanium dioxide per 1 part of glycerol, 85%), and 0.10 kg ferric oxide yellow in glycerol 85% (1:2) in 0.5 kg purified water, and stirred to homogeneity. This mixture is used for several capsule loads.

(b) The mass, manufactured according to paragraph (a), is transferred into a capsule forming apparatus while cooling, and encapsulated with the composition mentioned above containing artesunate and piperaquine according to the rotary-die method after R. P. Scherer. The capsules are pre-dried in a rotation dryer (system Scherer) until sufficient solidity is reached. Thereafter the capsules are further dried on racks. The incoming air is at 15 to 30° C. with 10-50% relative humidity. After drying faulty capsules are eliminated.

(c) For the coating of the capsules with a lubricant a perforated drum coating apparatus (Glatt GC) is used. The lacquer formulation is sprayed with forced incoming and outgoing air until the required amount is applied per capsule. For this purpose 0.9 kg polyethylene glycol with an average molecular weight 20000, 0.40 kg polyethylene glycyol with an average molecular weight of 1550, 0.60 kg glycerol monooleate and -dioleate (mixture), 0.04 kg polyvinyl acetate, and 0.30 kg talcum are dissolved or suspended, respectively, in an ethanol-water mixture per coating batch. The coated capsules are dried and inspected. Faulty capsules are eliminated. The coated capsules are then sealed in PVDC aluminium blister packs.

### EXAMPLE 2

### Manufacture of a Composition According to the Invention With Double the Amount of Piperaquine

40 kg triglycerides with intermediate chain length, 20 kg artesunate and 72 kg piperaquine tetraphosphate are intensively mixed and then treated further as in Example 1. For the gelatin capsules 55 kg gelatin, 22 kg glycerol 85% and 1.1 kg titanium dioxide, but no ferric oxide are used. For the coating of the capsules 1.24 kg polyethylene glycol 20000, 0.56 polyethylene glycol 1550, 0.82 of a mixture of glycerol monooleate and -dioleate, 0.06 kg polyvinyl acetate and 0.42 kg talcum are used.

1. Pharmaceutical composition for rectal use comprising artemisinine or a derivative of artemisinine and piperaquine or a pharmaceutically acceptable salt thereof.

2. Pharmaceutical composition according to claim 1 comprising arteether, artemether, artemisinine or artesunate, and piperaquine or a pharmaceutically acceptable salt thereof.

3. Pharmaceutical composition according to claim 1 comprising artesunate and piperaquine or a pharmaceutically acceptable salt thereof.

4. Pharmaceutical composition according to claim 1 comprising artesunate as the free acid and piperaquine as the tetraphosphate.

5. Pharmaceutical composition according to claim 1 in form of rectal capsule.

6. Pharmaceutical composition according to claim 5 wherein the rectal capsule consists of hard gelatin or soft gelatin and is supplied with a lubricating coat.

7. Pharmaceutical composition according to claim 3, characterized in that the weight proportion of artesunate to piperaquine is between 1:0.5 and 1:10.

8. Pharmaceutical composition according to claim 7, characterized in that the weight proportion of artesunate to piperaquine is between 1:0.5 and 1:2.5.

9. Pharmaceutical composition according to claim 7 comprising between 10 mg and 600 mg of a mixture of artesunate and piperaquine.

10. A method of treating malaria in children comprising administering to a patient in need thereof a therapeutically effective amount of a pharmaceutical composition for rectal use comprising artemisinine or a derivative of artemisinine and piperaquine or a pharmaceutically acceptable salt thereof.
